(19) United States
(12) Patent Application Publication
(10) Pub. No.: US 2023/0364196 A1
(43) Pub. Date: Nov. 16, 2023

(54) METHODS FOR TREATING GRAFT-VERSUS-HOST DISEASE USING GLP-2 AGONISTS AND ANALOGUES THEREOF

(71) Applicant: THE TRUSTEES OF COLUMBIA UNIVERSITY IN THE CITY OF NEW YORK, New York, NY (US)

(72) Inventors: Ran Reshef, New York, NY (US); David Harle, New York, NY (US)

(21) Appl. No.: 18/223,327

(22) Filed: Jul. 18, 2023

Related U.S. Application Data
(63) Continuation of application No. PCT/US2022/013075, filed on Jan. 20, 2022.
(60) Provisional application No. 63/139,490, filed on Jan. 20, 2021.

(51) Int. Cl. A61K 38/26; A61K 35/28; A61P 37/06; A61K 45/06

(52) U.S. Cl. CPC A61K 38/26; A61K 35/28; A61P 37/06; A61K 45/06

(57) ABSTRACT

The present invention provides, inter alia, methods for systemically treating or preventing graft-versus-host disease (GVHD) in a subject using a GLP-2 analogue or GLP-2 agonist. Also provided are methods for treating an autoimmune disorder in a subject, methods for improving the effect of a cancer treatment in a subject in need thereof, methods for systemically treating an inflammatory condition in a subject caused by solid organ transplant rejection, and methods for reducing high-dose chemotherapy- and/or radiotherapy-induced GI mucositis, using a GLP-2 analogue or GLP-2 agonist.

### CROSS REFERENCE TO RELATED APPLICATIONS

The present application is a continuation of PCT international application no. PCT/US2022/013075, filed on Jan. 20, 2022, which claims benefit of U.S. Provisional Patent Application Ser. No. 63/139,490, filed on Jan. 20, 2021. The entire contents of the aforementioned applications are incorporated by reference as if recited in full herein.

### GOVERNMENT FUNDING

This invention was made with government support under W81XWH-19-1-0578 awarded by Army/MRMC. The government has certain rights in the invention.

### FIELD OF THE INVENTION

The present invention relates to methods for systemically treating or preventing immune-mediated systemic inflammatory disorders including, e.g., autoimmune diseases such as IBD, graft-versus-host disease (GVHD), and gut microbiome related diseases, in a subject using a GLP-2 analogue or GLP-2 agonist.

### BACKGROUND OF THE INVENTION

Glucagon-like peptide-2 (GLP-2) is an important neuroendocrine mediator that acts as an enterocyte-specific growth hormone. GLP-2 induces enterocyte proliferation, prevents apoptosis, enhances the mucosal barrier and enhances nutrient absorption (Drucker et al. 1996; Munroe et al. 1999). GLP-2 also plays a role in the interaction between gut epithelium and the microbiome (Cani et al. 2009). GLP-2 is produced by neuroendocrine cells in the ileum and colon. In animal models of severe enteropathies, GLP-2 analogues have been shown to reverse loss of enterocyte mass, increase nutrient absorption, decrease intestinal inflammation and reduce bacterial translocation.

The role of GLP-2 analogues in hematopoietic stem-cell transplantation has not been examined in experimental models. The effect of GLP-2 analogues on gut mucosae has been examined in animals receiving standard doses of chemotherapy and was examined in patients undergoing standard doses of chemotherapy with the goal of developing this drug as a supportive care measure against chemotherapy-induced diarrhea. In clinical trials, these drugs have been given at a schedule that initiates drug treatment before chemotherapy which was thought to maximize the treatment effect.

The role of GLP-2 analogues in immune-mediated systemic inflammatory disorders (including graft-versus-host disease and systemic autoimmune disorders) has not been examined.

More than 30,000 allogeneic transplants are performed each year worldwide for a wide range of hematologic malignancies and benign disorders and the number is growing steadily. Graft-versus-host disease (GVHD) remains the most common cause of treatment-related morbidity and mortality after allogeneic stem-cell transplantation. Gut GVHD is the most common form of severe GVHD. Moreover, the mucosal immune system of the gut is thought to be a critical site in the initiation of systemic GVHD. The neuroendocrine axis that regulates diverse gut function, including nutrient absorption, motility, inflammation and most importantly enterocyte proliferation and tissue repair, has not been explored in the setting of allogeneic stem-cell transplantation and its potential effects on conditioning-induced mucositis, loss of microbial diversity and decreased systemic immune activation are unknown. Current approaches for GVHD are either highly immunosuppressive and increase risk for lethal infections, or increase cancer relapse due to loss of the graft-versus-tumor effect.

### SUMMARY OF THE INVENTION

In the present disclosure, it is believed that GLP-2 can have a positive clinical effect on maintenance of gut epithelium, recovery of the gut from damage by chemotherapy and radiotherapy related to to a specific timing of exposure to GLP-2, diversity and composition of gut microbiome, translocation of microbes and microbial products, and can also have a beneficial therapeutic effect against immune activation, including but not limited to the setting of alloreactivity experienced after allogeneic hematopoietic stem-cell transplantation. In this setting, GLP-2 has a protective effect against graft-versus-host disease (GVHD) after allogeneic hematopoietic stem-cell transplantation that can be used for therapeutic benefit in humans, leading to decreased rates of GVHD after transplant, decreased use of immunosuppressive agents after transplant, decreased rate of infections by gut bacteria after transplant and improved survival of transplant patients.

Accordingly, in the present disclosure, it is believed that GLP-2 analogues have a protective role against gut mucosal damage and GVHD in allogeneic hematopoietic stem-cell transplant patients through three potential mechanisms—1) by attenuating tissue damage induced by transplant conditioning and preserving the mucosal barrier thereby preventing translocation of bacterial products that activate immune cells and stimulate systemic alloreactivity, 2) by reducing the inflammatory response induced in the gut by alloreactive T-cells, pro-inflammatory macrophages and dendritic cells, and promoting effective tissue repair, and 3) by altering the microbiome after high dose chemotherapy and/or radiation in a way that creates a more favorable immune environment.

In the present disclosure, GLP-2 agonists can be used in the prevention and treatment of GVHD, and potentially used in other diseases where gut inflammation is thought to be the culprit, including inflammatory bowel disease and other autoimmune disorders that are provoked by activation of mucosal immune cells by the content of the gut.

Additionally, GLP-2 analogues can be used in treating or preventing gut mucositis after total body radiotherapy or high-dose chemotherapy that are unique to transplant conditioning regimens, interventions that are more potent and lead to more aggressive mucosal damage compared to standard doses of chemotherapies that have been tested previously. Some of the chemotherapies used for transplant conditioning (e.g., melphalan) cause severe gut mucositis which is dose limiting due to toxicity. Radiation also causes severe mucosal damage which limits its dose. Decreasing gut mucositis or improving tissue repair may allow administration of higher doses of chemotherapy or radiation than currently feasible, thereby potentially improving cancer control and survival of cancer patients. It may also allow combination therapies for transplant conditioning that are currently not feasible due to excess gut epithelial toxicity. Reduction in mucositis may also improve quality of life of cancer patients undergoing high-dose chemotherapy or radiation or allogeneic transplant patients who are all at risk for GVHD, decrease infections, hospitalizations and healthcare resource utilization.

In the present disclosure, it is also believed that the administration schedule currently used by clinicians (i.e., initiating GLP-2 stimulation prior to chemotherapy) may impair the treatment effect by further sensitizing the intestinal stem cells to the effect of radiotherapy or chemotherapy, thereby negating the desired therapeutic effect.

GLP-2 analogues or GLP-2 agonists represent an innovative approach to modulating the mucosal immune system in the GI tract, the gut microbiome and attenuating systemic immune responses and can be particularly useful in GVHD prevention and treatment. The potential applications of the present disclosure may also include other areas of therapy where high-dose chemotherapy and/or radiation are used such as autologous stem-cell transplantation and in situations where radiation is used in the abdominal area where radiation dose to the bowel is high and causes mucositis and radiation-induced enteritis and colitis.

The receptor that is stimulated by GLP-2 analogues or GLP-2 agonists is also expressed in lung tissue and therefore a similar favorable modulation of the immune system and the local microbiome is expected with therapeutic use of GLP-2 analogues or GLP-2 agonists. Therefore, these agents can be used in immune-mediated diseases of the lung, including GVHD, radiation pneumonitis and inflammation resulting from autoimmune diseases or lung infections.

The mechanism by which GLP-2 analogues and GLP-2 agonists exert a favorable effect on the immune system in the mucosae and elsewhere is through an increase in tolerogenic macrophages and dendritic cells.

Thus, one embodiment of the present disclosure is a method for systemically treating or preventing graft-versus-host disease (GVHD) in a subject. This method comprises administering to the subject a therapeutically effective amount of a GLP-2 analogue or GLP-2 agonist.

As used herein, a “GLP-2 agonist” refers to an agent that simulates GLP-2 itself or the GLP-2 receptor via any mechanism.

Another embodiment of the present disclosure is a method for treating an immune-mediated systemic inflammatory disorder in a subject. This method comprises administering to the subject a therapeutically effective amount of a GLP-2 analogue or GLP-2 agonist.

Another embodiment of the present disclosure is a method for improving the effect of a cancer treatment in a subject in need thereof. This method comprises administering to the subject a therapeutically effective amount of a GLP-2 analogue or GLP-2 agonist.

Still another embodiment of the present disclosure is a method for systemically treating an inflammatory condition in a subject caused by solid organ transplant rejection. This method comprises administering to the subject a therapeutically effective amount of a GLP-2 analogue or GLP-2 agonist.

Yet another embodiment of the present disclosure is a method for reducing high-dose chemotherapy- and/or radiotherapy-induced GI mucositis. This method comprises administering to a subject in need thereof an effective amount of a GLP-2 analogue or GLP-2 agonist, wherein the GLP-2 analogue or GLP-2 agonist is administered after completion of the chemotherapy and/or radiotherapy.

A further embodiment of the present disclosure is a method for modulating gut microbiome in a subject in need thereof. This method comprises administering to the subject a therapeutically effective amount of a GLP-2 analogue or GLP-2 agonist.

Another embodiment of the present disclosure is a method for enhancing the innate immune system in a subject. This method comprises administering to the subject a therapeutically effective amount of a GLP-2 analogue or GLP-2 agonist.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following drawings form part of the present specification and are included to further demonstrate certain aspects of the present invention. The invention may be better understood by reference to one or more of these drawings in combination with the detailed description of specific embodiments presented herein.

FIG. 1 shows the body weight change of BALB/c syngeneic transplant recipients after high-dose lethal radiation upon different treatment schedules with elsiglutide, a GLP-2 analogue.

FIG. 2 shows the bowel weight change of BALB/c syngeneic transplant recipients after high-dose lethal radiation upon different treatment schedules with elsiglutide.

FIG. 3A shows the histological result of elsigltuide treatment on the distal small bowel of BALB/c syngeneic transplant recipients after high-dose lethal radiation.

FIG. 3B shows the histological result of elsigltuide treatment on the distal small bowel of BALB/c syngeneic transplant recipients after high-dose lethal radiation (magnified 20 times).

FIG. 4 shows the result of TUNEL stain performed to assess apoptosis of intestinal epithelial cells on D4 after high-dose lethal radiation.

FIG. 5 shows that higher doses of elsiglutide enhanced the total body weight of healthy mice (unirradiated).

FIG. 6 shows that elsiglutide treatment enhanced the mass of the entire intestine.

FIG. 7 shows the results of Ki67 proliferation staining in healthy mice and elsiglutide-treated mice.

FIG. 8 shows the effect of different elsiglutide treatment schedules on animal weight and graft-versus-host disease score in major MHC-mismatched (allogeneic) murine hematopoietic stem-cell transplantation.

FIG. 9 shows the treatment effect on intestinal mass.

FIG. 10 shows the results of the treatment effect of elsiglutide administered prior to and after major MHC-mismatched (allogeneic) murine hematopoietic stem-cell transplantation.

FIG. 11 shows the results of the treatment effect of elsiglutide after discontinuing treatment.

FIG. 12 shows comparable engraftment in the spleen of CD45+ hematopoietic cells, CD3+ T-cells and their subsets CD4 and CD8 cells and engraftment of myeloid cells after treatmented with elsigltide vs. vehicle control.

FIG. 13 shows the absence of weight loss and even increase in small bowel mass and length in elsiglutide-treated mice.

FIG. 14 shows that elisglutide-treated mice show fewer T-EMRA cells in both CD4 and CD8 T-cells and in both intraepithelial and lamina propria lymphocytes (IEL and LPL) compared to vehicle-treated mice.

FIG. 15 shows higher CD44 expression on gut T-cells in elsiglutide-treated mice.

FIG. 16 shows lower CD103 expression in donor CD4+ populations after elsiglutide treatment in MHC-mismatched allogeneic transplant.

FIG. 17 shows that treatment with GLP-2 reduces the relative abundance of Tbet⁺ intraepithelial CD4⁺ and CD8⁺ T-Cells following allogeneic transplantation. BALB/C mice underwent lethal TBI. (850 cGy Cs137) and transplantation with 5×10⁶ T-cell depleted bone marrow cells and 1×10⁶ splenic T-cells from B6 donors (major MHC-mismatch model). Daily subcutaneous administration of 800 nmol/kg Elsiglutide (GLP-2) reduced the relative fraction of Tbet⁺ T-cells within the intraepithelial compartment of the small intestine, suggesting a reduced pro-inflammatory phenotype.

FIG. 18 shows the differences in T-cell infiltration in the colon of elsiglutide-treated vs vehicle-treated mice.

FIG. 19 shows the differences in T-cell infiltration in the small bowel of elsiglutide-treated vs vehicle-treated mice.

FIG. 20 shows the macroscopic assessment (1×) of lung infiltration.

FIG. 21 shows the lung infiltrates at 20× magnification.

FIG. 22 shows the alveolar diameter and alveolar wall thickness in elsiglutide-treated vs vehicle-treated mice.

FIG. 23 shows decreased inflammation in the liver portal triads in elsiglutide-treated mice compared to vehicle-treated mice (20× magnification). The yellow arrows point at the inflammatory infiltrate in the portal triads in the vehicle-treated mice.

FIG. 24 shows the assessment of serum cytokine levels from mice undergoing allogeneic transplantation via LegendPlex. The results revealed reduced serum IFNg on D+21 post-transplant, suggesting a lower systemic inflammatory state.

FIGS. 25A-25C show that GLP-2 treatment leads to large shifts in lamina propria mononuclear-phagocytes following allogeneic transplantation. FIG. 25A shows that daily subcutaneous treatment with Elsiglutide (GLP-2) in allogeneically transplanted BALB/C mice (B6 donor) led to the increased prevalence of lamina propria macrophages expressing markers associated with maturation (increased MHC II and CX3CR1, reduced Ly6C) on D+21 post-transplant. FIG. 25B shows that these macrophages had reduced expression of co-stimulatory molecules and increased SIRPα (inhibitor of phagocytosis) suggestive of a more tolerogenic phenotype associated with mature lamina propria macrophages. tSNE plots were generated on CD45⁺CD19⁻CD3⁻Ly6G-SSC^{low/int} cells that expressed either CD11b or CD11c (or both) with the plots above representing a merge of 10 mice (n=5 for vehicle, 5 for GLP-2). FIG. 25C shows significant reduction in Ly6C^{High} MHC-II^{Low} immature macrophages.

FIG. 26 shows that GLP-2 treatment appears to enhance the maturation of lamina propria macrophages on D+7 following syngeneic transplantation. To assess the impact of GLP-2 on macrophage maturation in the absence of inflammatory GVHD, BALB/C mice were transplanted with T-cell depleted bone marrow from BALB/C donors and given daily S.C. injections of GLP-2 as mentioned above. Assessment of lamina propria macrophages on D+7 revealed an increased shift toward mature “P4” macrophages in GLP-2 treated mice, suggesting GLP-2R stimulation promotes maturation of recruited monocytes. P1-P4 macrophages were gated as CD45+CD19⁻CD3-Ly6G⁻ and CD11b⁺ or CD11c⁺. Subsets of the “monocyte-waterfall” were gated as: P1: CD64^{-/low} Ly6C⁺MHC II⁻ CX3CR1^{-/low}; P2: CD64^{low/+}Ly6C⁺ MHC II⁺ CX₃CR1^{-/low}; P3: CD64⁺ Ly6C⁻MHC II⁺ CX3CR1^{-/low}; P4: CD64⁺Ly6C⁻MHC II⁺ CX3CR1⁺. N=4 per treatment group.

FIG. 27 shows that Treatment with GLP-2 alters lamina propria innate lymphoid cells (ILCs) in the syngeneic but not allogeneic setting on D+14 post-transplant. Daily subcutaneous injection of GLP-2 increases the relative abundance of lamina propria ILCs in a process that appears to target type-3 ILCs (ILC3s). ILC3s are important immunomodulators of the lamina propria and intestinal stem-cell (ISC) niche via their production of IL-22, playing a role in ISC maintenance. Importantly, treatment with GLP-2 in the allogeneic setting did not enhance recovery of any ILC subtype, suggesting the protective mechanism that GLP-2 confers against GVHD is not dependent on ILCs (which are thought to be targeted by alloreactive cells in GVHD). ILCs were gated as CD45⁺ CD19⁻ CD3⁻ Ly6G⁻ CD11b⁻ CD11c⁻ CD127⁺, with subsets being assigned based on Tbet (ILC1), GATA-3 (ILC2) and RORgt (ILC3) expression.

FIGS. 28A-28B show that GLP-2 treatment impacts microbial shifts following lethal TBI and syngeneic transplantation. Mice undergoing lethal TBI were treated with daily S.C. injections with Elsiglutide (GLP-2) and their stool collected for 16s rRNA sequencing on D+0 (baseline), D+14 and D+28 post-transplant. Treatment with GLP-2 appeared to ameliorate the impact of lethal TBI on the intestinal microbiota, where β-diversity plots (FIG. 28A) showed overlap between D+0 and D+14 samples. Vehicle treated mice, conversely, demonstrated distinct diversity clusters. Importantly, continual treatment with GLP-2 resulted in shifts in β-diversity by D+28, however this clustering was distinct from vehicle-treated animals. Relative abundance plots (FIG. 28B) suggested that Akkermansia muciniphila, a mucous degrading microbe, was increased in GLP-2 mice, particularly on D+14.

FIG. 28C shows cage conditions experiment to examine the role the microbiota may play in GVHD. BALB/C mice underwent lethal TBI and allogeneic transplantation were assigned to 3 cage conditions to perturb their microbial composition: 1) vehicle and GLP-2 treated animals housed together, 2) treatment groups housed separately or 3) treatment groups housed separately and provided with neomycin and polymyxin-b in their drinking water from D-2 to D+14 post-transplant. Survival curves were impacted by the various caging conditions, with vehicle-treated animals demonstrating increased survival when housed with their GLP-2 treated counterparts. Conversely, disruption of the intestinal microbiota via antibiotics reduced the efficacy of GLP-2 treatment. Together, these results suggest that shifts in microbial communities occur due to GLP-2 treatment and that these shifts play an essential role in the beneficial impact of GLP-2 on GVHD.

### DETAILED DESCRIPTION OF THE INVENTION

One embodiment of the present disclosure is a method for systemically treating or preventing graft-versus-host disease (GVHD) in a subject. This method comprises administering to the subject a therapeutically effective amount of a GLP-2 agonist or analogue thereof.

As used herein, a “graft-versus-host disease” or “GVHD” is a condition that might occur after an allogeneic transplant. In GVHD, the donated bone marrow or peripheral blood stem cells view the recipient's body as foreign, and the donated cells/bone marrow attack the body. Non-limiting exemplary target organs of GVHD include skin, liver, upper and lower GI, and lung. In some embodiments, the GVHD is acute or chronic GVHD.

In the present disclosure, an “effective amount” or “therapeutically effective amount” of an agent or pharmaceutical composition is an amount of such an agent or composition that is sufficient to affect beneficial or desired results as described herein when administered to a subject. Effective dosage forms, modes of administration, and dosage amounts may be determined empirically, and making such determinations is within the skill of the art. It is understood by those skilled in the art that the dosage amount will vary with the route of administration, the rate of excretion, the duration of the treatment, the identity of any other drugs being administered, the age, size, and species of the subject, and like factors well known in the arts of, e.g., medicine and veterinary medicine. In general, a suitable dose of an agent or pharmaceutical composition according to the disclosure will be that amount of the agent or composition, which is the lowest dose effective to produce the desired effect with no or minimal side effects. The effective dose of an agent or pharmaceutical composition according to the present disclosure may be administered as two, three, four, five, six or more sub-doses, administered separately at appropriate intervals throughout the day.

In some embodiments, the subject received allogeneic hematopoietic stem-cell transplantation (HSCT).

As used herein, a “subject” is a mammal, preferably, a human. In addition to humans, categories of mammals within the scope of the present invention include, for example, agricultural animals, veterinary animals, laboratory animals, etc. Some examples of agricultural animals include cows, pigs, horses, goats, etc. Some examples of veterinary animals include dogs, cats, etc. Some examples of laboratory animals include primates, rats, mice, rabbits, guinea pigs, etc.

In some embodiments, the GLP-2 analogue is selected from the group consisting of human [Gly²] GLP-2, glepaglutide, NM-003, teduglutide, apraglutide, and elsiglutide. In some embodiments, the GLP-2 analogue is elsiglutide. In the present disclosure, pharmaceutically acceptable salts of the GLP-2 analogues or GLP-2 agonists are also included. Pharmaceutical compositions of any of the foregoing are also contemplated by the present disclosure.

In some embodiments, the methods disclosed herein further comprise co-administering to the subject an immunosuppressive agent. As used herein, an “immunosuppressive drug”, “immunosuppressive agent” or “antirejection medication” refers to an agent that inhibits or prevents activity of the immune system. Non-limiting examples of immunosuppressive agents include prednisone (Deltasone, Orasone), budesonide (Entocort EC), prednisolone (Millipred), tofacitinib (Xeljanz), cyclosporine (Neoral, Sandimmune, SangCya), tacrolimus (Astagraf XL, Envarsus XR, Prograf), sirolimus (Rapamune), everolimus (Afinitor, Zortress), azathioprine (Azasan, Imuran), leflunomide (Arava), mycophenolate (CellCept, Myfortic), abatacept (Orencia), adalimumab (Humira), anakinra (Kineret), certolizumab (Cimzia), etanercept (Enbrel), golimumab (Simponi), infliximab (Remicade), ixekizumab (Taltz), natalizumab (Tysabri), rituximab (Rituxan), secukinumab (Cosentyx), tocilizumab (Actemra), ustekinumab (Stelara), vedolizumab (Entyvio), basiliximab (Simulect), daclizumab (Zinbryta), muromonab (Orthoclone OKT3), antithymocyte globulin (Thymoglobulin, Atgam, Grafalon), alemtuzumab (Campath, Lemtrada), ruxolitinib, itacitinib, and combinations thereof.

Another embodiment of the present disclosure is a method for treating an immune-mediated systemic inflammatory disorder in a subject. This method comprises administering to the subject a therapeutically effective amount of a GLP-2 analogue or GLP-2 agonist. Non-limiting examples of immune-mediated systemic inflammatory disorders include multiple sclerosis, rheumatoid arthritis, solid organ transplant rejection, autoimmune hepatitis, nonalcoholic steatohepatitis, celiac disease, inflammatory bowel disease, food allergies, and asthma. In some embodiments, the autoimmune disorder is inflammatory bowel disease (IBD).

Another embodiment of the present disclosure is a method for improving the effect of a cancer treatment in a subject in need thereof. This method comprises administering to the subject a therapeutically effective amount of a GLP-2 analogue or GLP-2 agonist.

In some embodiments, the administration of a GLP-2 analogue or GLP-2 agonist is after the subject receives the cancer treatment regimen. In certain embodiments, the GLP-2 analogue or GLP-2 agonist is administered to the subject from seconds, to hours, to weeks post-cancer treatment, for example, from 1 to 120 minutes post-cancer treatment, from 1 to 30 days post-cancer treatment or from 1 to 10 weeks post-cancer treatment.

In some embodiments, the cancer treatment regimen is selected from chemotherapy, radiotherapy, immunotherapy, autologous transplant, allogeneic transplant, and combinations thereof.

In some embodiments, the chemotherapy comprises co-administering to the subject a chemotherapy drug selected from the group consisting of cisplatin, temozolomide, doxorubicin, cyclophosphamide, methotrexate, 5-fluorouracil, vinorelbine, docetaxel, bleomycin, vinblastine, dacarbazine, mustine, melphalan, vincristine, procarbazine, prednisolone, etoposide, epirubicin, capecitabine, methotrexate, folinic acid, oxaliplatin, fludarabine, busulfan, clofarabine, and combinations thereof.

In some embodiments, the cancer treatment regimen is allogeneic hematopoietic stem-cell transplantation (HSCT).

In some embodiments, the improvement of effect includes lower gut epithelial toxicity of the cancer treatment.

Still another embodiment of the present disclosure is a method for systemically treating an inflammatory condition in a subject caused by solid organ transplant rejection. This method comprises administering to the subject a therapeutically effective amount of a GLP-2 analogue or GLP-2 agonist.

Yet another embodiment of the present disclosure is a method for reducing high-dose chemotherapy- and/or radiotherapy-induced GI mucositis. This method comprises administering to a subject in need thereof an effective amount of a GLP-2 analogue or GLP-2 agonist, wherein the GLP-2 analogue or GLP-2 agonist is administered after completion of the chemotherapy and/or radiotherapy.

A further embodiment of the present disclosure is a method for modulating gut microbiome in a subject in need thereof. This method comprises administering to the subject a therapeutically effective amount of a GLP-2 analogue or GLP-2 agonist. In some embodiments, the subject has a disease that can be therapeutically beneficial from the modulation of gut microbiome.

Another embodiment of the present disclosure is a method for enhancing the innate immune system in a subject. This method comprises administering to the subject a therapeutically effective amount of a GLP-2 analogue or GLP-2 agonist.

In some embodiments, the subject has an immune-mediated systemic inflammatory disorder.

In some embodiments, enhancing the innate immune system comprises recovering homeostasis of an innate immune cell. In some embodiments, the innate immune cell is selected from the group consisting of a macrophage, a dendritic cell, an innate lymphoid cell, and combinations thereof.

### EXAMPLES

The following examples are provided to further illustrate certain aspects of the present disclosure. These examples are illustrative only and are not intended to limit the scope of the disclosure in any way.

### Example 1

### Effect of GLP-2 Stimulation on Healthy Mice and Mice Treated with High-Dose Radiation

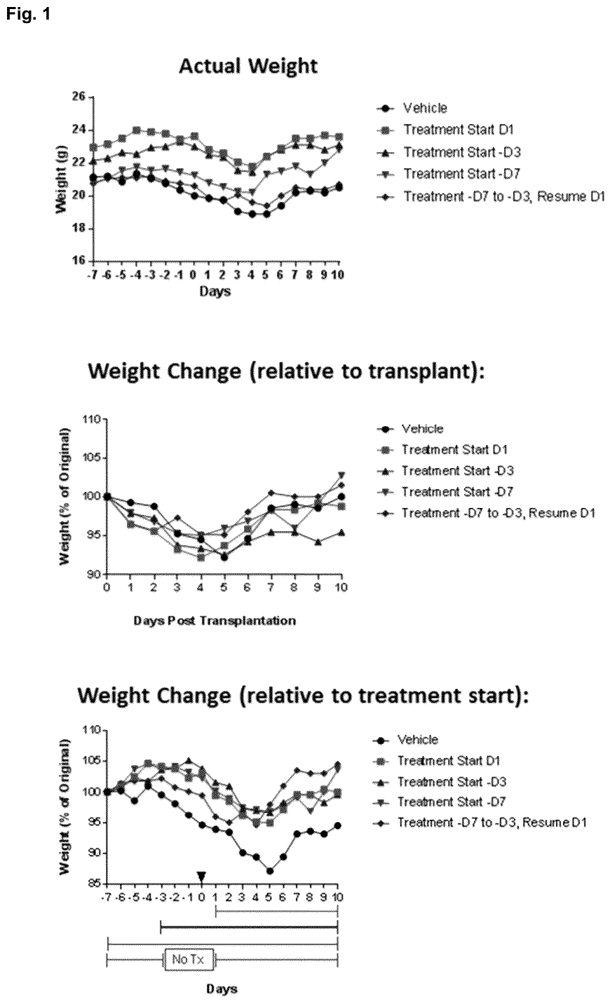

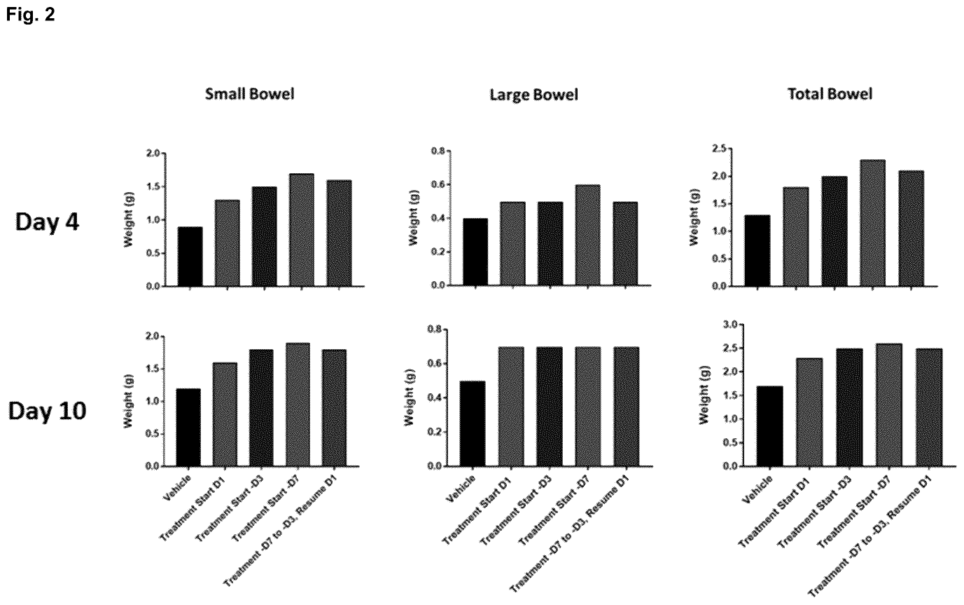

This experiment was carried out to determine the effect of daily elsiglutide administration (800 nmol/kg) on weight recovery and bowel weight in response to high dose radiotherapy using different treatment schedules. Syngeneic transplant was performed to rescue the animals from early death due to hematopoietic failure to be able to examine the mice at several time points. Several treatment schedules were examined here and as expected, mice recovered weight after high dose radiotherapy without a clear effect of elsiglutide treatment. Treatment start on D-3 performed slightly worse than other schedules in terms of weight recovery after radiation (FIG. 1). Treatment with elsiglutide increased small bowel mass after irradiation and syngeneic transplant. The longer the treatment the more pronounced the effect. There was a similar albeit reduced effect in the large bowel (FIG. 2).

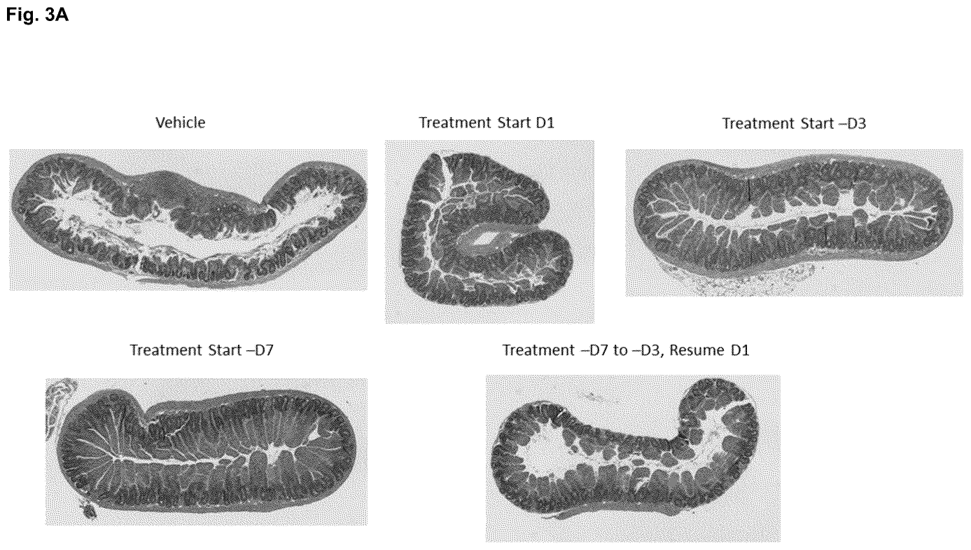

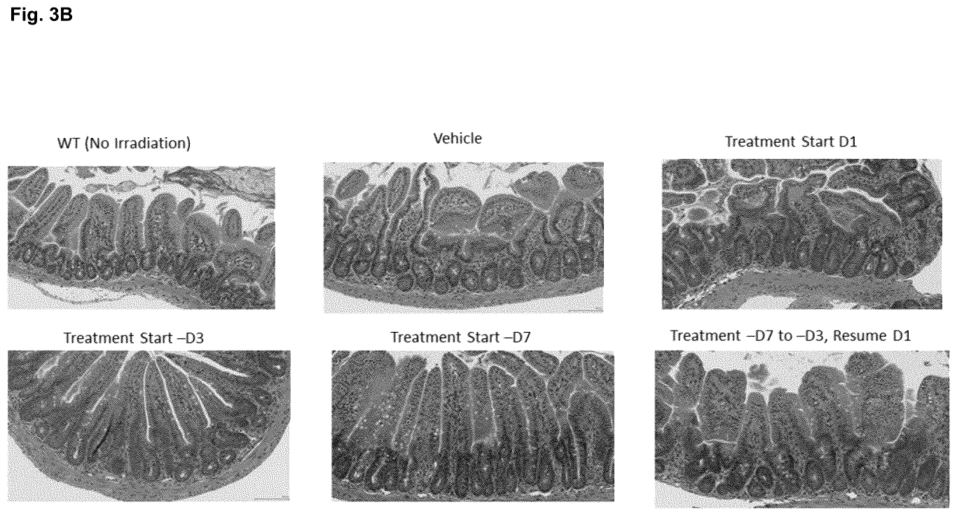

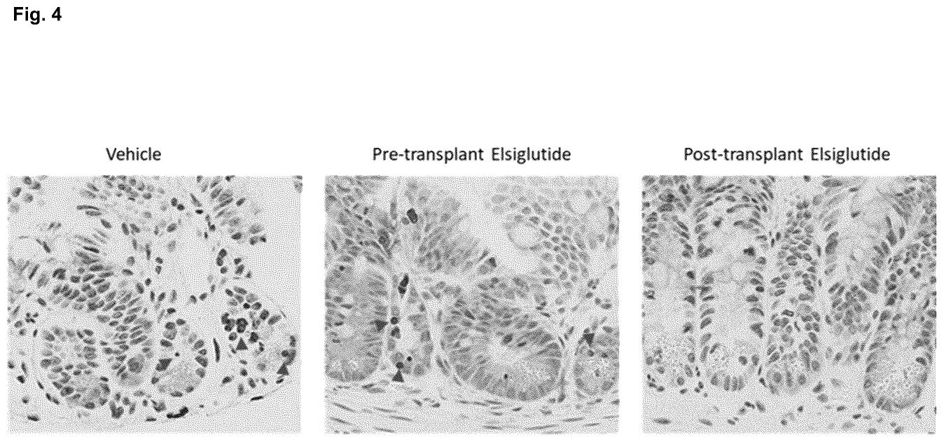

Histologically, elsigltuide treatment enhanced villi length and crypt depth after radiation with the longer treatment courses associated with the most pronounced effect (FIGS. 3A and 3B). TUNEL stain was then performed to assess apoptosis of intestinal epithelial cells on D4 after radiation. We observed a small number of apoptotic bodies within the crypts of all mice at D4 post irradiation and transplantation (FIG. 4). Surprisingly, pre-transplant treated animals (D-7) showed increased abundance of apoptotic bodies within the crypts compared to vehicle treated mice, implying that elsiglutide treatment sensitized the epithelium to radiation damage and potentially worsened the epithelial damage. Animals beginning treatment D1 post-transplant had almost no apoptotic bodies present within the crypts. Animals that received treatment from D-7 to D-3 then resumed on D1 (so elsiglutide was not present during irradiation) also had very few apoptotic bodies (similar to the post-transplant group).

We conclude that the administration of elsiglutide during the radiation process may be detrimental to the GI mucosa and impair the ability of the mucosae to recover. An optimal treatment schedule might be to start elsiglutide after radiation.

### Example 2

### Impact of Different Elsiglutide Doses on the Total Body and Bowel Weight of Healthy BALB/c Mice (No Irradiation or Transplant)

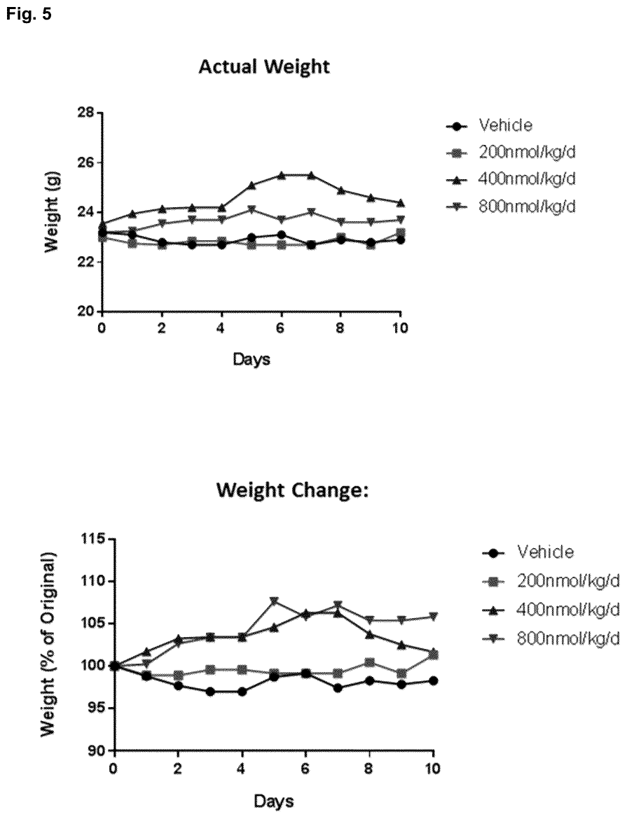

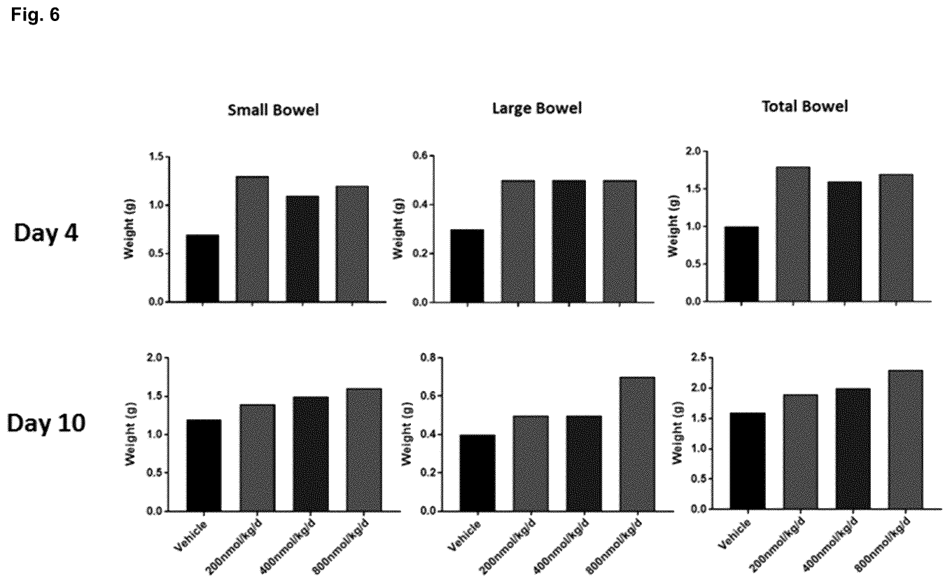

In this experiment elsiglutide at escalating doses (200-800 nmol/kg/d) was injected to healthy mice without irradiation or chemotherapy for 10 days. We observed a dose-dependent weight increase over baseline (FIG. 5) and a dose-dependent increase in the weight of both small and large bowel in these mice by day 10 (FIG. 6).

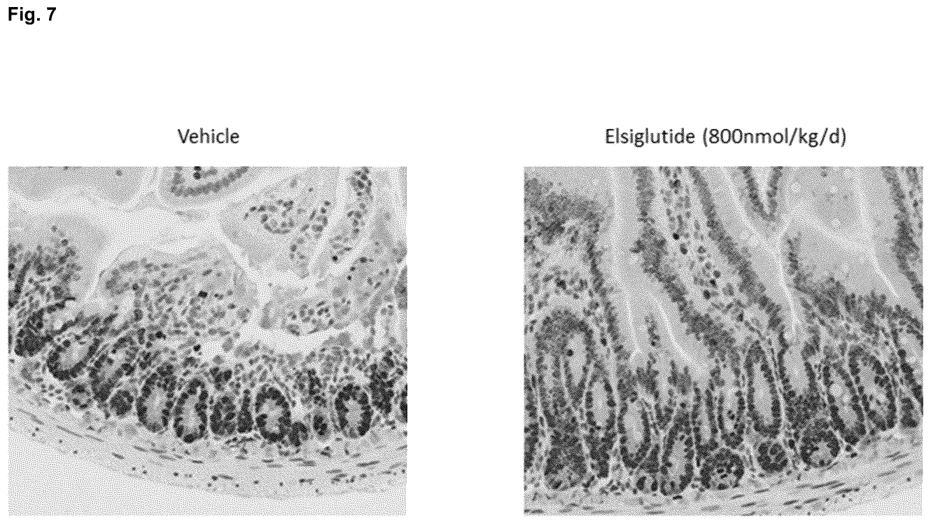

In this experiment we also performed Ki67 stains to document the proliferation of crypt cells in response to elsiglutide (FIG. 7). In healthy mice treated with vehicle Ki67 immunoreactivity was predominantly confined to the crypts where the stem cells reside. In elsiglutide-treated animals Ki67 staining extended further out of the crypts into the villi, potentially reflecting the expansion and growth seen on the macroscopic level. There was no obvious difference between the different doses in this assay.

### Example 3

### GLP-2 Stimulation in Prevention of GVHD after Allogeneic HSCT

To determine the effect of elsiglutide treatment on graft-versus-host disease in MHC-mismatched (allogeneic) murine hematopoietic stem-cell transplantation and identify the optimal dosing schedule, in this experiment BALB/c mice were lethally-irradiated and transplanted with bone marrow (5×10⁶ cells) from B6 mice plus 2×10⁶ splenic T-cells and treated with 800 nmol/kg/d elsiglutide at 4 different treatment schedules (syngeneic transplant and allogeneic transplant with vehicle were used as controls).

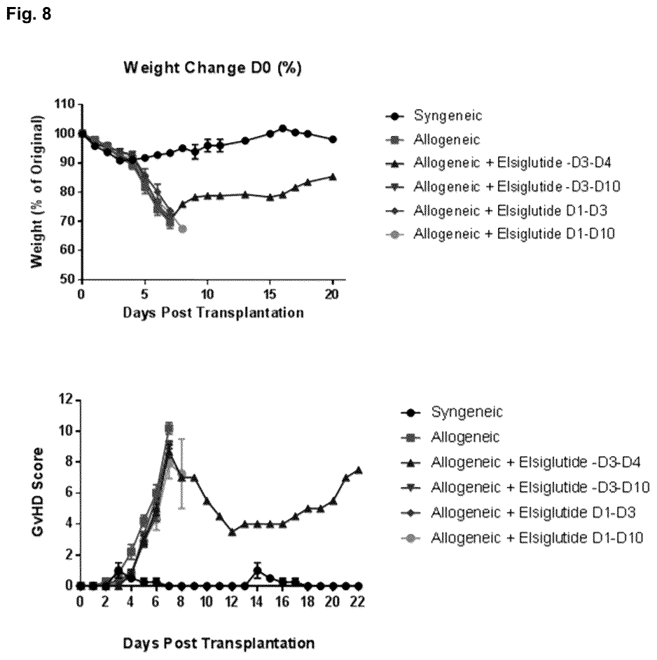

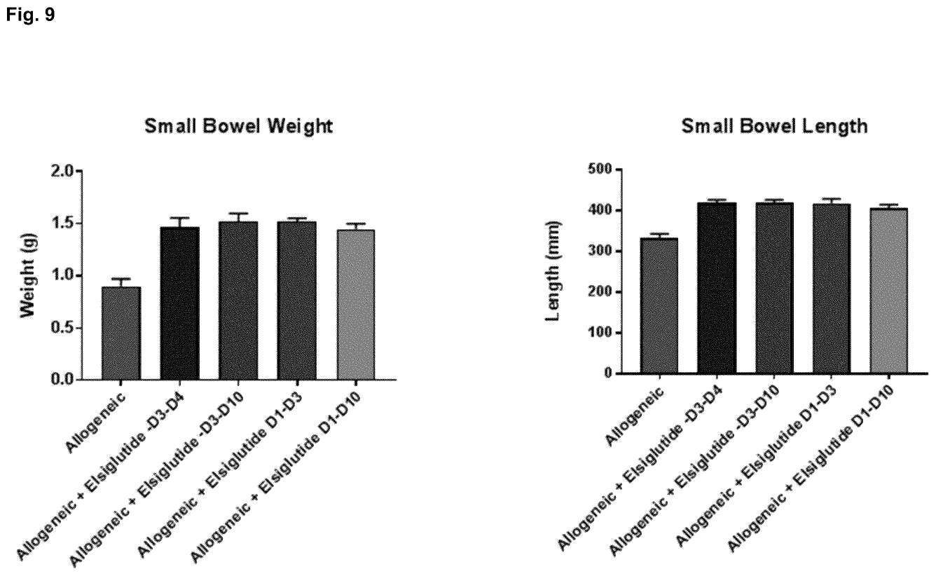

In this experiment, we observed an increase in small bowel weight and length similar to what we observed in syngeneic transplants. All mice except 1 from −D3-D4 group developed severe GvHD by D6 and had to be euthanized by D8. The one surviving mouse partially recovered and then died on D22 (FIG. 8). We hypothesized that this may be due to the 2×10⁶ T-cell dose which is considered the upper-limit and causes severe disease that is too extreme for treatment to overcome. The treatment effect on intestinal mass was observed (FIG. 9). This was statistically significant for both weight and length (p<0.0001) and was not dependent on treatment schedule.

### Example 4

### GLP-2 Stimulation in Prevention of GVHD after Allogeneic HSCT (with Lower T-Cell Dose)

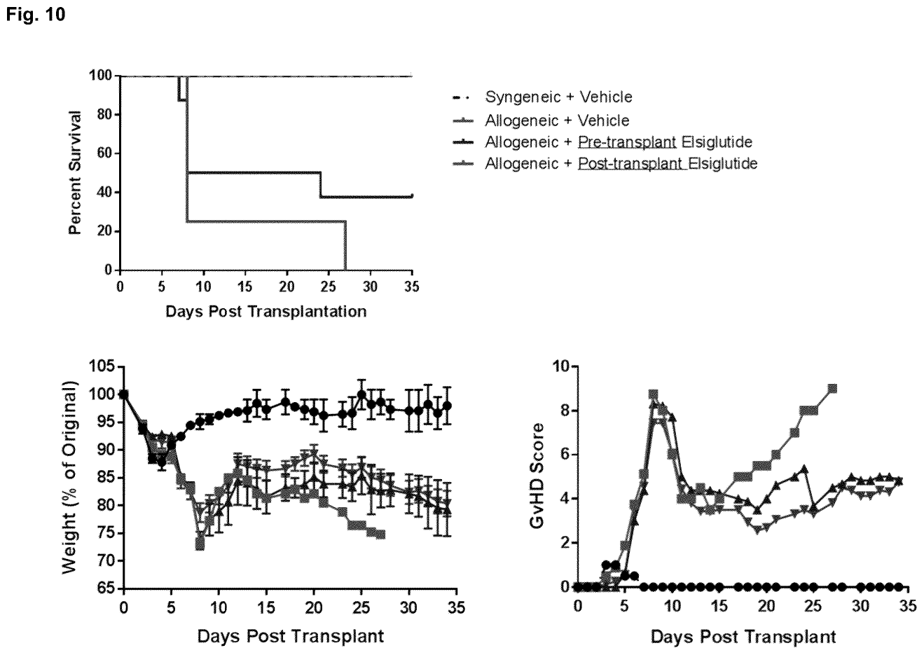

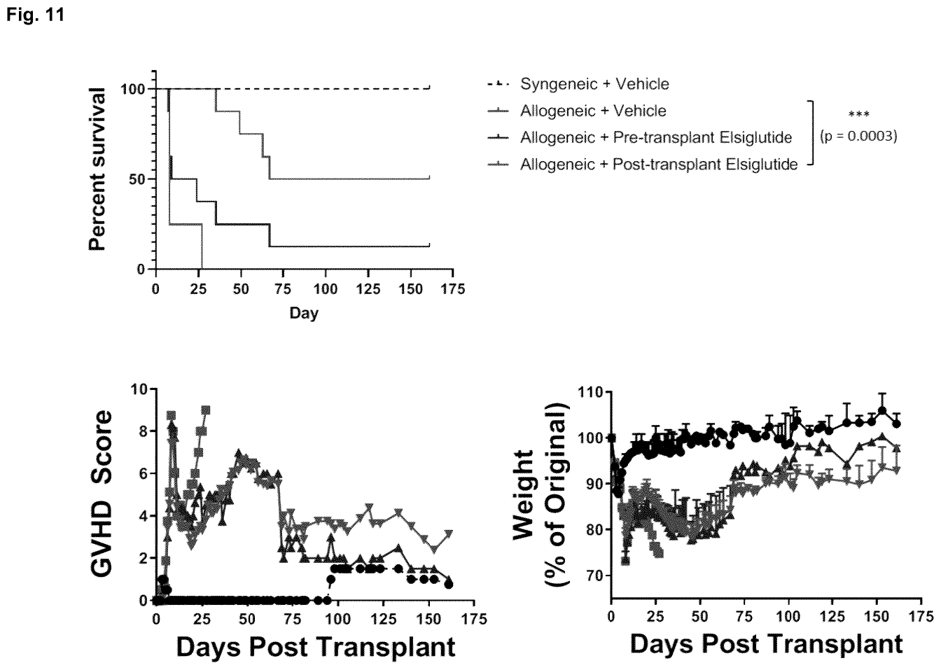

In a subsequent experiment, we used the same design as Example 3 but with T-cell depleted (TCD) bone marrow (BM) cells (5×10⁶ cells) and a lower T-cell dose (0.5×10⁶ splenic T cells) to allow examination of survival differences in a major MHC-mismatched GVHD model (aggressive GVHD model). As treatment effect was similar in this experiment for groups 3-4 and 5-6, these groups were combined into an “elsiglutide pre-transplant” group and an “elsiglutide post-transplant” group for presentation. In addition, animals originally designated to end treatment on D4 resumed treatment on D9 and continued until D30 or SAC. This time, the administration of elsiglutide beginning on D1 post-transplantation rescued 100% of mice from lethal GVHD along with improvement in GVHD scores and weight. Pre-transplant elsiglutide still had an effect on GVHD scores and weight in surviving mice but rescued only 40% of mice from death through day 35. Control mice (allogeneic transplant with vehicle control) had 100% mortality as expected and syngeneic control mice had 100% survival as expected (FIG. 10). This further supports the hypothesis that the timing of administration of the drug (post- vs. pre-radiation conditioning) is critical.

After discontinuation of treatment (day 30), the treatment effect persisted and 50% of mice in the post-transplant elsiglutide group survived long term (FIG. 11). This shows that the treatment effect modulates the immune system and leads to immune tolerance in a very aggressive model. This also shows that the effect of elsiglutide is systemic and affects GVHD in target organs outside the GI tract.

### Example 5

### Mechanistic Studies of GLP-2 Analogues and their Activities within and Outside the GI System

To further explore the mechanism behind the treatment effect observed in Example 4, we used the same experimental design as Example 4. In this experiment, mice underwent a major MHC mismatched allogeneic transplant were treated with post-transplant elsiglutide (Group 3) vs. vehicle (Group 2) (TCD BM only (no GVHD) as Group 1) and sacrificed on D7 and their organs (spleen, MLN, GI tract, liver, lungs) were extracted for flow cytometry/histology.

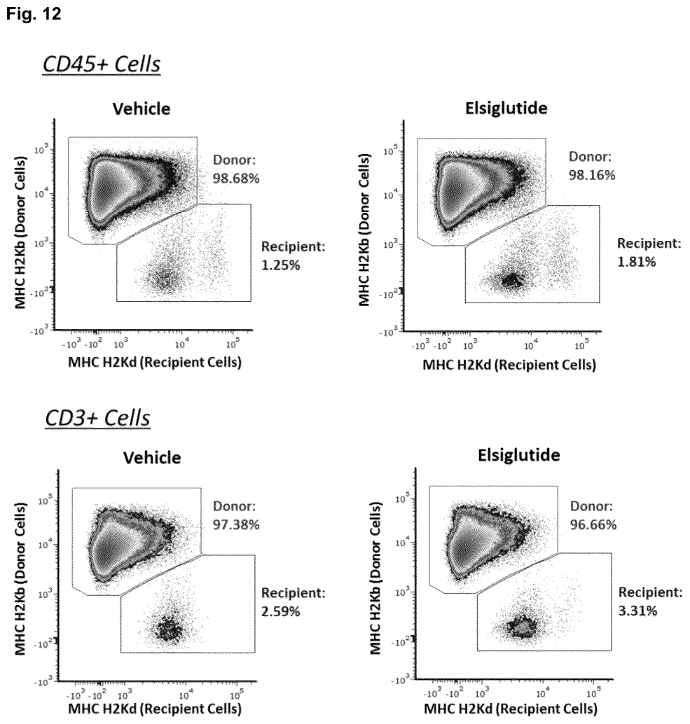

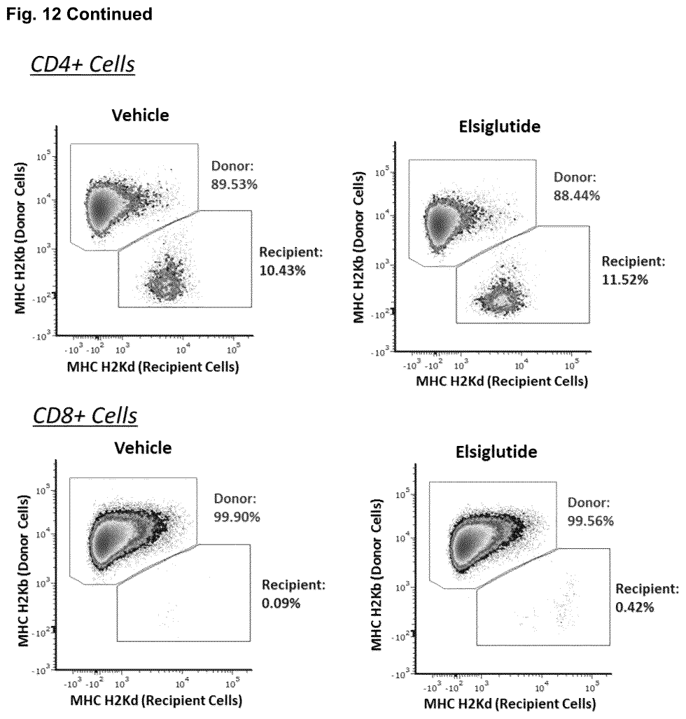

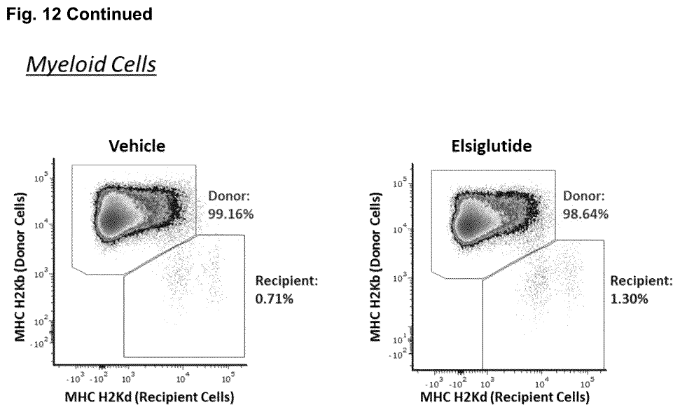

As shown in FIG. 12, elsiglutide treatment did not impair engraftment of hematopoietic stem cells. Mice allografted from a major MHC mismatched donor demonstrated donor cell engraftment in the spleen at similar levels for elsiglutide-treated mice vs. vehicle controls. These findings demonstrated the safety of administering elsiglutide in allogeneic HCT recipients without impairing the transplant process.

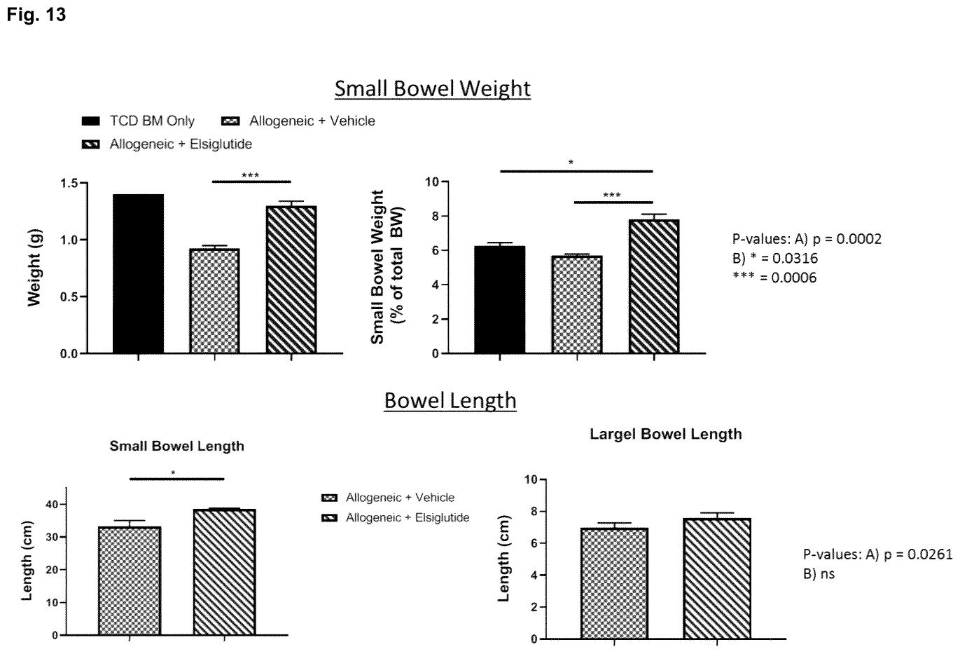

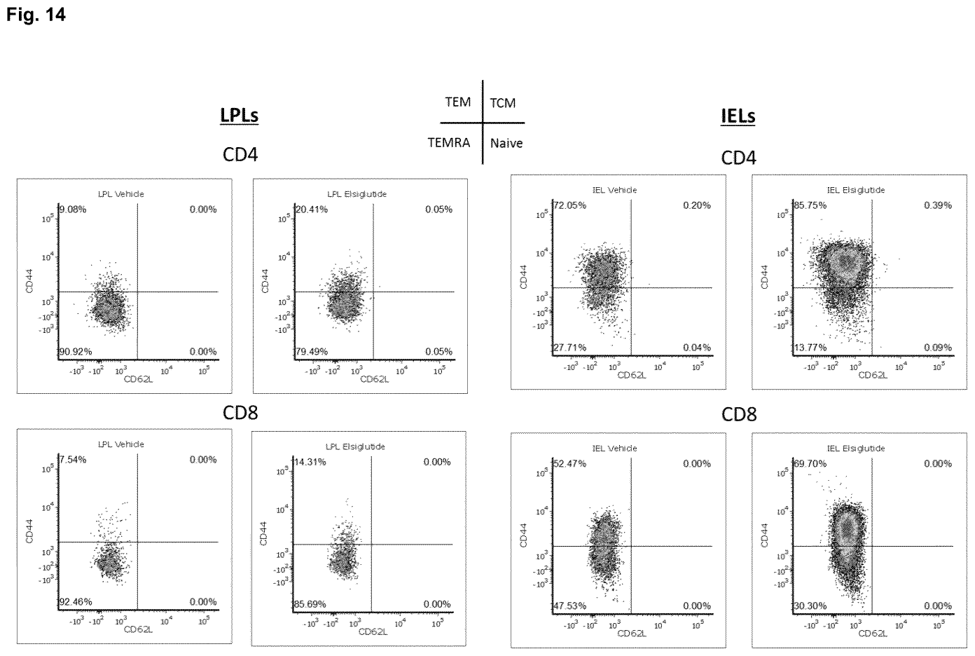

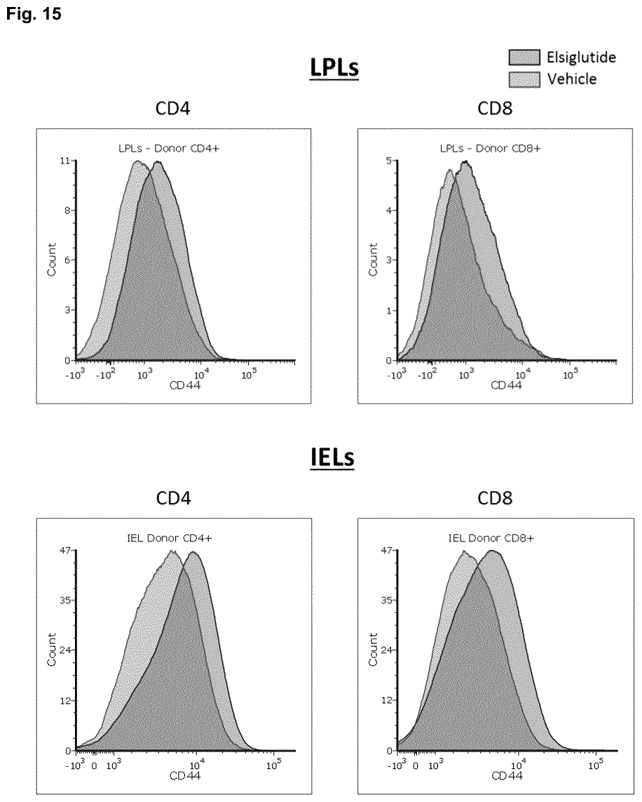

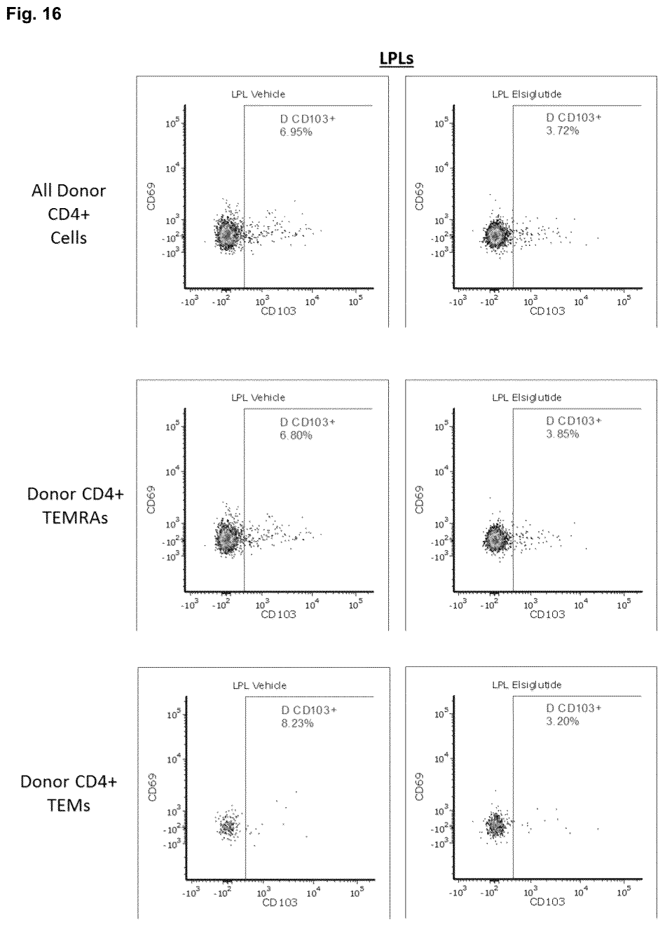

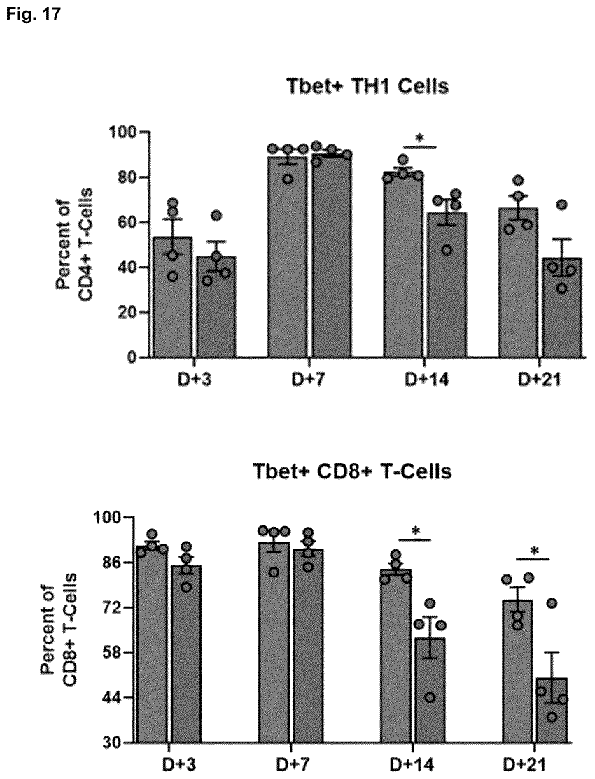

With respect to the observations from the gut, elsiglutide treatment rescued mice from the damaging effects of GVHD, as demonstrated in absence of weight loss and even increase in small bowel mass and length in elsiglutide-treated mice (FIG. 13). Elsiglutide treatment reduced T-cell activation on day 7 after transplant. Treated mice showed fewer T-EMRA cells in both CD4 and CD8 T-cells and in both intraepithelial and lamina propria lymphocytes (IEL and LPL) compared to vehicle-treated mice (FIG. 14), which were consistent with fewer activated cells that have undergone terminal differentiation. As shown in FIG. 15, higher CD44 expression on gut T-cells in elsiglutide-treated mice demonstrated an increase in donor T-Effector Memory cells on the expense of T-EMRA terminally differentiated cells, and implied a reduced level of allogeneic immune activation. In conclusion, elsiglutide treatment decreased level of expression of adhesion molecules on gut lamina propria T-cells. CD103 expression on CD4 T-cells was shown as a representative example (FIG. 16). Treatment with elsiglutide also resulted in a lower proportion of Th1 and CD8+ T-cells expressing the transcription factor T-bet, which further supports a less activated function (FIG. 17).

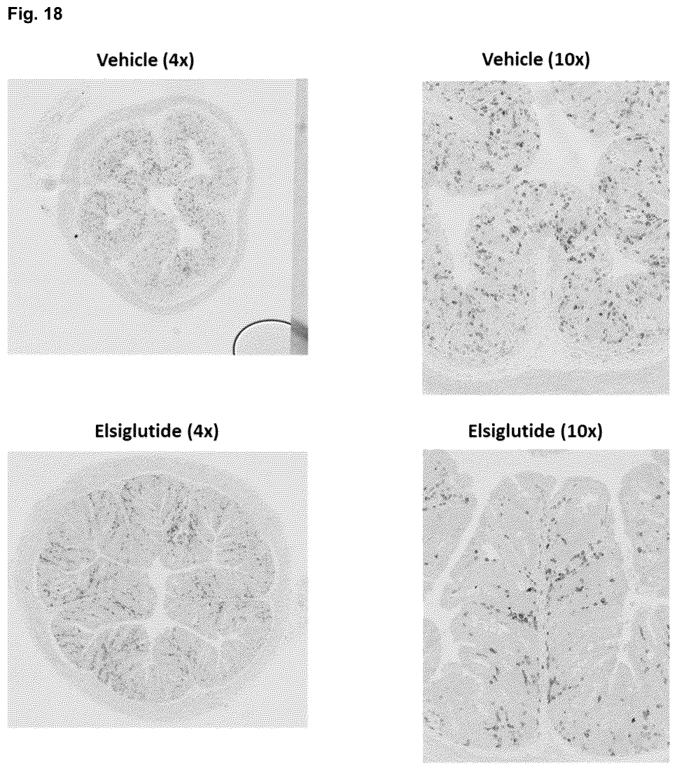

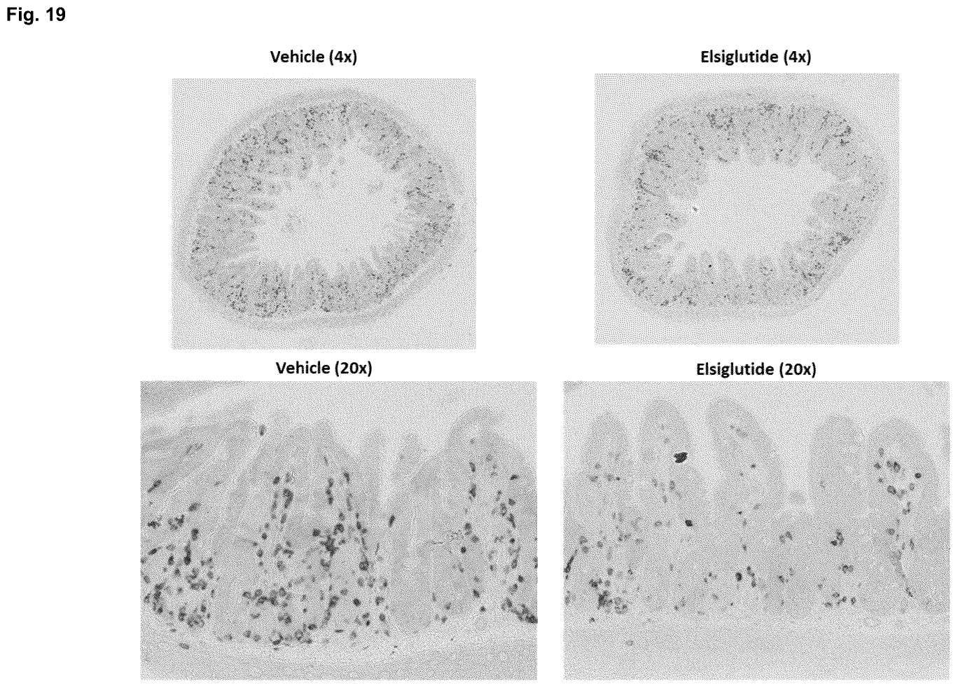

We examined the T-cell infiltration in the colon and the small bowel, and found that immunohistochemistry for CD3+ T-cells showed differences in T-cell infiltration. Elsiglutide-treated mice showed decreased T-cell infiltration with more focal and less diffuse pattern of infiltration and fewer intraepithelial T-cells compared to vehicle treated mice (FIG. 18 and FIG. 19).

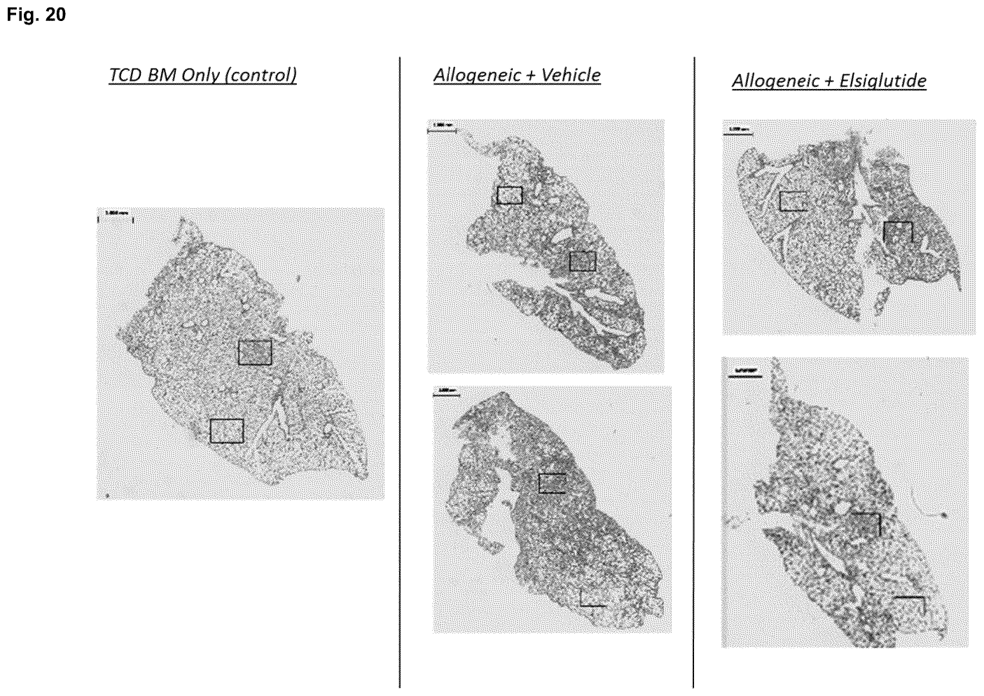

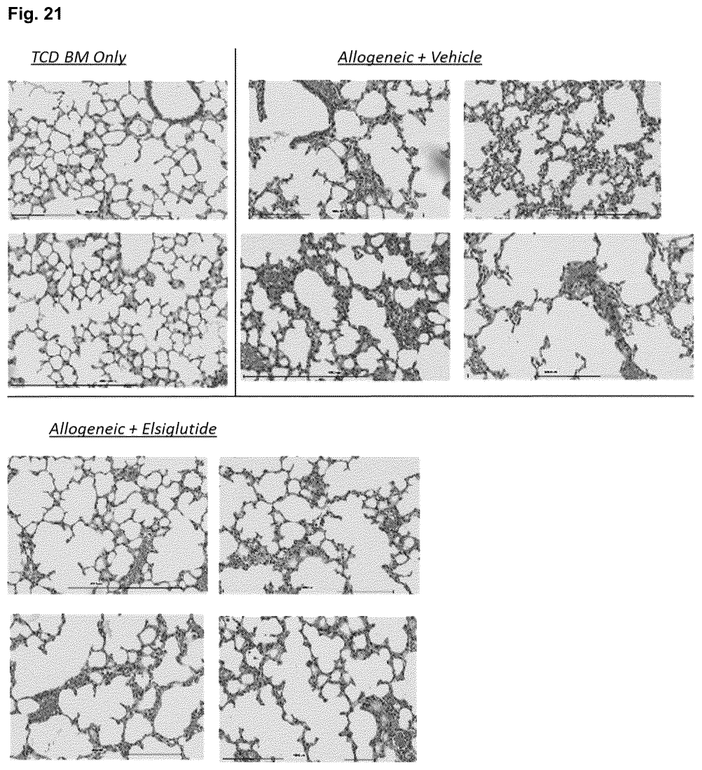

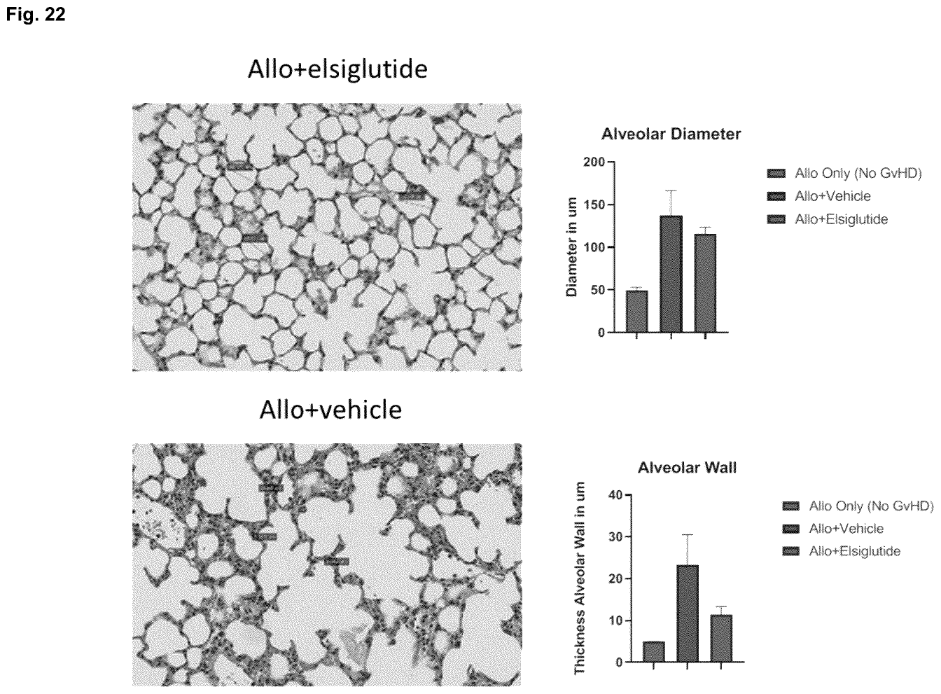

We also found that increased cell infiltration and inflammation in vehicle-treated mice illustrated the anti-inflammatory effect of elsiglutide outside the GI tract. Treated mice showed fewer areas of inflammation and healthier appearing lung tissue compared to allogeneic transplant mice with vehicle control (FIG. 20). Treated mice had decreased infiltration and less alveolar wall thickening, and the lung appearance was similar to controls (FIG. 21). Alveolar diameter and alveolar wall thickness in elsiglutide-treated mice showed decreased inflammation and alveolar damage in the lung compared to vehicle-treated mice (FIG. 22).

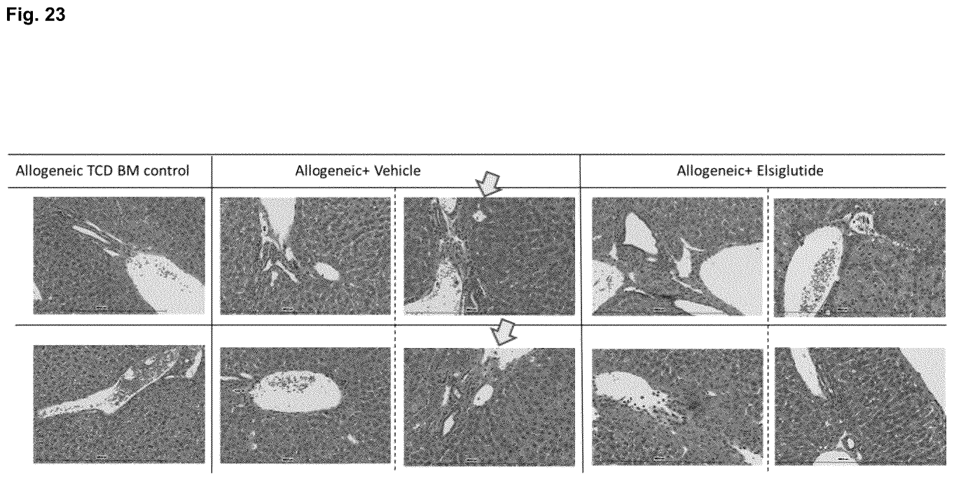

Besides lung, elsiglutide-treated allogeneic transplanted mice also showed decreased inflammation in the liver portal triads compared to vehicle-treated mice (FIG. 23), demonstrating again the protective effect of elsiglutide on GVHD target organs outside the GI tract, and showing a systemic anti-inflammatory effect.

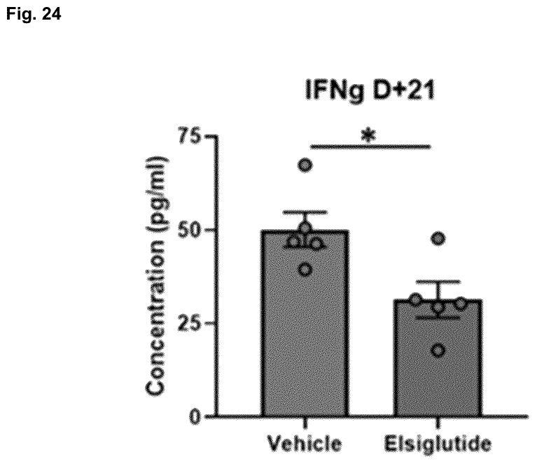

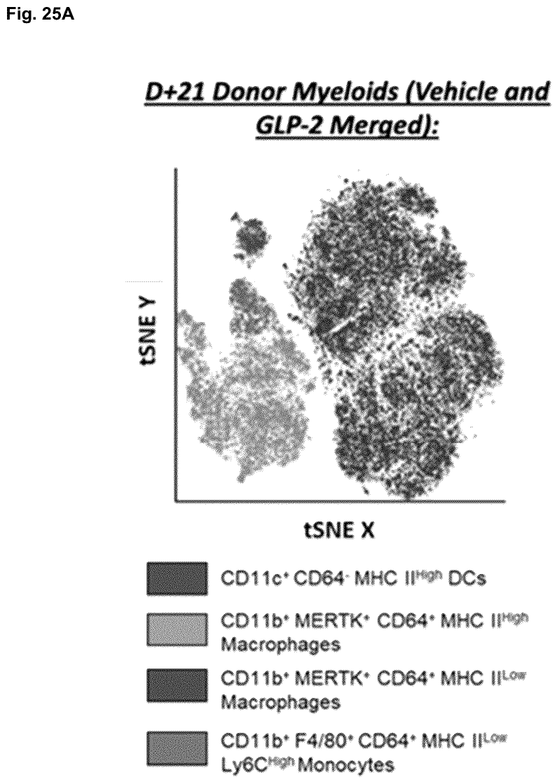

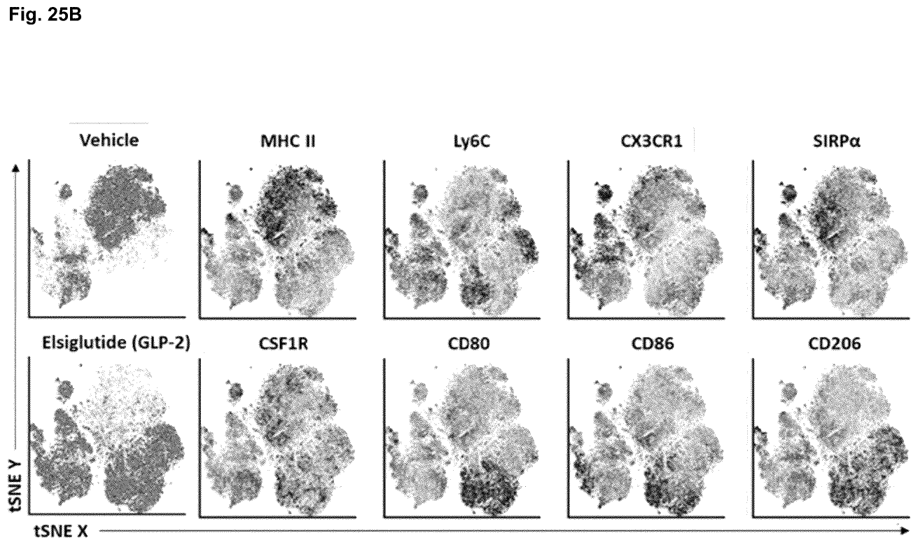

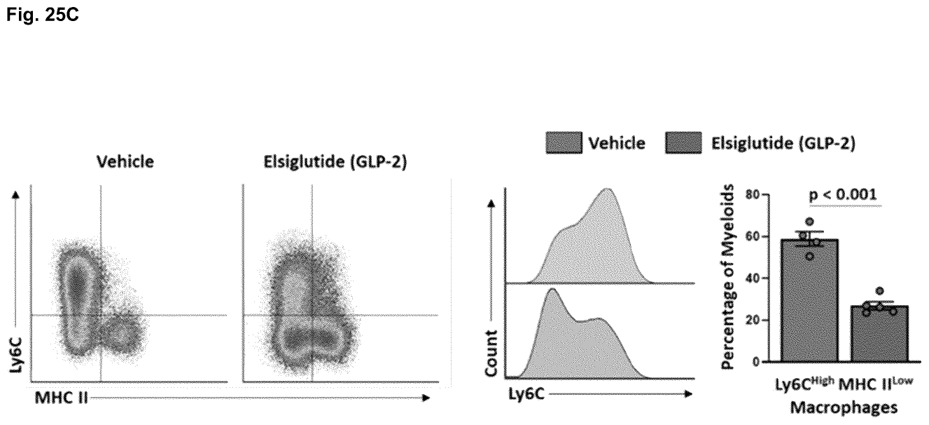

In further support of a decrease in systemic inflammation, elsiglutide-treated transplanted mice had lower levels of interferon gamma in the serum on D+21 after transplant compared to vehicle-treated mice (FIG. 24). In the same GVHD model (major MHC-mismatched transplant, as described in Example 4), we identified that the primary driver for the protective effect against gut mucosal inflammation was a change in the phenotype of macrophages in the gut lamina propria. As illustrated in FIGS. 25A-25C, elsiglutide treatment was associated with an increase in lamina propria macrophages that had a tolerogenic phenotype—higher expression of CX3CR1 and SIRP-alpha, but lower expression of the costimulatory molecules CD80 and CD86 and the phagocytic marker CD206. Elsiglutide treatment was also associated with a decrease in Ly6C^{high}MHC-II^{low} macrophages, again consistent with a recovery of healthy and tolerogenic homeostasis of innate immune cells in the gut mucosae.

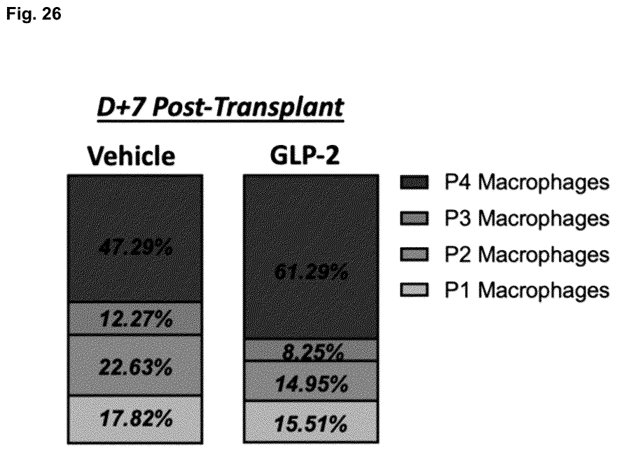

Changes in the phenotype of innate immune cells were also demonstrated in the non-allogeneic HCT setting. In a syngeneic transplant (similar to Example 1), which simulates severe GI damage from radiation but without alloreactivity, there was improved recovery of mature macrophages (also known as P4 macrophages) in elsiglutide-treated mice compared to vehicle-treated mice (FIG. 26) as early as D+7 post-transplant.

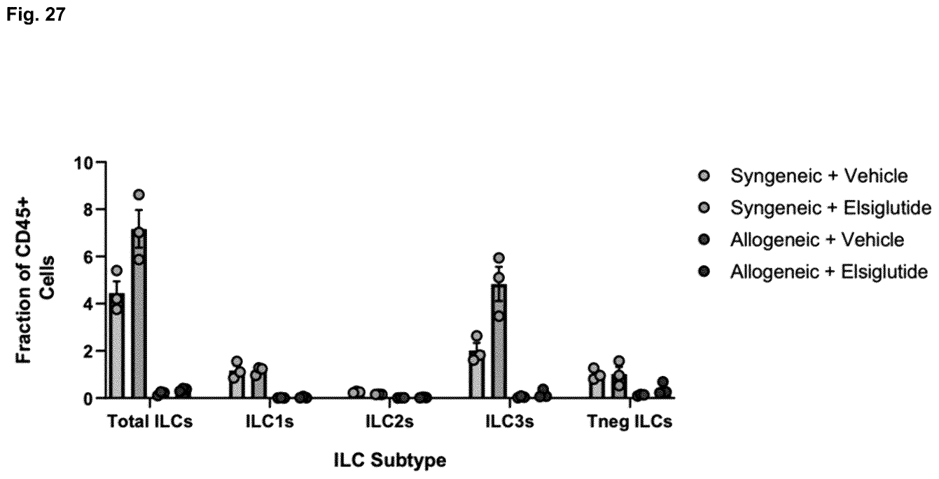

Elsigutide treatment also had an impact on innate lymphoid cells (ILCs), further supporting the mechanism of action of the drug by impacting innate immune homeostasis (FIG. 27). In a syngeneic transplant model (similar to example 1), recovery of ILCs after transplant was improved in elsiglutide-treated mice and this improvement was driven by a significant increase in ILC3, which stabilize the intestinal barrier, assist in gut recovery from radiation damage, regulate the gut microbiome, and generate IL-22 which protects the intestinal epithelium.

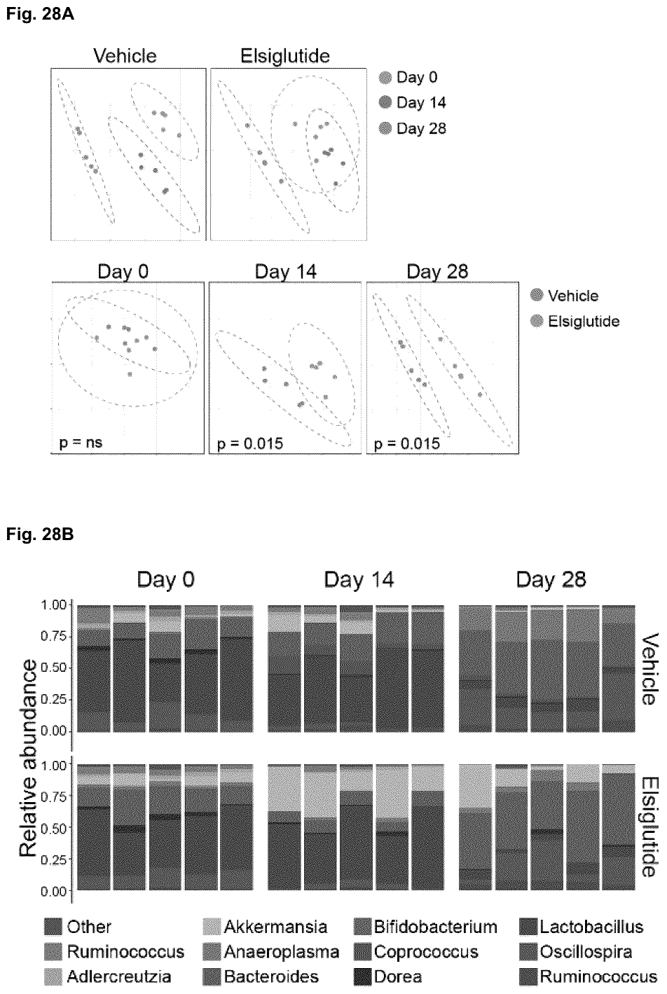

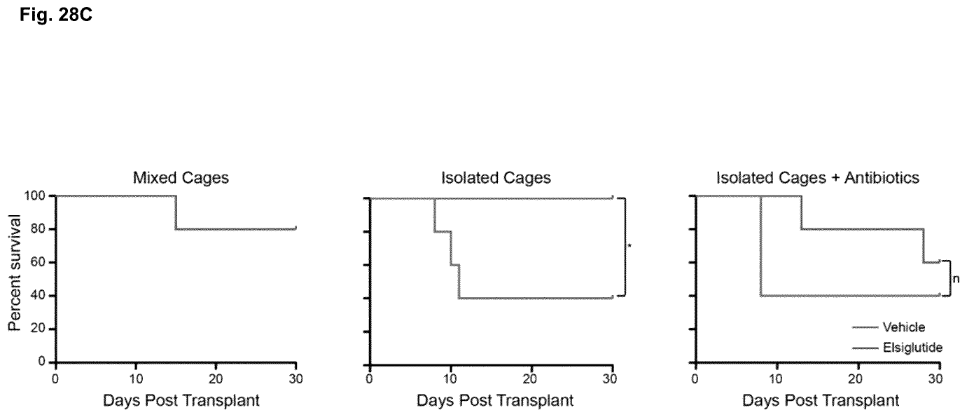

To assess potential mechanisms for the differences in macrophage and T-cell phenotype, we examined the impact of GLP-2 on the intestinal microbiota. A syngeneic BALB/cJ model (similar to Example 1) was used to explore the effects of GLP-2 independent of GVHD. Stool samples from D+0, D+14, and D+28 were subjected to 16S rRNA sequencing. Vehicle-treated mice had distinct p-diversity clusters at all time-points, showing a transplant effect on the microbiota (FIG. 28A). GLP-2-treated mice had near-complete cluster overlap between D+0 and D+14, suggesting attenuation of the impact of conditioning. GLP-2 treated mice were significantly enriched for Akkermansia muciniphila and Bacteroidales S24-7 family at D+14 and D+28 (FIG. 28B). These taxa have been associated with anti-inflammatory properties and A. muciniphila abundance is linked to epithelial mucin production, which is increased by GLP-2.

We then assessed the role of microbial communities in the protective effect of GLP-2 by conducting an allogeneic transplant with 3 caging conditions; 1) vehicle and GLP-2 treated mice caged together, 2) caged separately, or 3) caged separately plus oral antibiotics. We observed a clear cage effect where co-housing the treatment groups improved the survival of vehicle treated mice (FIG. 28C), suggesting transferal of the therapeutic effect via the microbiome. Antibiotic administration also dampened the beneficial effect of GLP-2.

### DOCUMENTS CITED

1 Cani P D, Possemiers S, Van de Wiele T, Guiot Y, Everard A, Rottier O, Geurts L, Naslain D, Neyrinck A, Lambert D M, Muccioli G G, Delzenne N M. Changes in gut microbiota control inflammation in obese mice through a mechanism involving GLP-2-driven improvement of gut permeability. Gut. 2009 August; 58(8): 1091-103.
2. Drucker D J, Asa S L, Brubaker P L. Induction of intestinal epithelial proliferation by glucagon-like peptide 2. PNAS. 1996 July; 93(15): pp. 7911-7916.
3. Munroe D G, Gupta A K, Kooshesh F, Vyas T B, Rizkalla G, Wang H, Demchyshyn L, Yang Z J, Kamboj R K, Chen H, McCallum K, Sumner-Smith M, Drucker D J, Crivici A. Prototypic G protein-coupled receptor for the intestinotrophic factor glucagon-like peptide 2. Proc Natl Acad Sci USA. 1999 Feb. 16; 96(4):1569-73.

All patents, patent applications, and publications cited above are incorporated herein by reference in their entirety as if recited in full herein.

The invention being thus described, it will be obvious that the same may be varied in many ways. Such variations are not to be regarded as a departure from the spirit and scope of the invention and all such modifications are intended to be included within the scope of the following claims.

What is claimed is:

1. A method for systemically treating or preventing graft-versus-host disease (GVHD) in a subject, comprising administering to the subject a therapeutically effective amount of a GLP-2 analogue or GLP-2 agonist.

2. The method of claim 1, wherein the GVHD is acute or chronic GVHD.

3. The method of claim 1, wherein the subject received allogeneic hematopoietic stem-cell transplantation (HSCT).

4. The method of claim 1, wherein the subject is a mammal.

5. The method of claim 4, wherein the mammal is selected from the group consisting of humans, veterinary animals, and agricultural animals.

6. The method of claim 1, wherein the subject is a human.

7. The method of claim 1, wherein the GLP-2 analogue is selected from the group consisting of human [Gly²] GLP-2, teduglutide, apraglutide, glepagultide, NM-003, and elsiglutide.

8. The method of claim 1, wherein the GLP-2 analogue is elsiglutide.

9. The method of claim 1, further comprising co-administering to the subject an immunosuppressive agent selected from the group consisting of prednisone (Deltasone, Orasone), budesonide (Entocort EC), prednisolone (Millipred), tofacitinib (Xeljanz), cyclosporine (Neoral, Sandimmune, SangCya), tacrolimus (Astagraf XL, Envarsus XR, Prograf), sirolimus (Rapamune), everolimus (Afinitor, Zortress), azathioprine (Azasan, Imuran), leflunomide (Arava), mycophenolate (CellCept, Myfortic), abatacept (Orencia), adalimumab (Humira), anakinra (Kineret), certolizumab (Cimzia), etanercept (Enbrel), golimumab (Simponi), infliximab (Remicade), ixekizumab (Taltz), natalizumab (Tysabri), rituximab (Rituxan), secukinumab (Cosentyx), tocilizumab (Actemra), ustekinumab (Stelara), vedolizumab (Entyvio), basiliximab (Simulect), daclizumab (Zinbryta), muromonab (Orthoclone OKT3), antithymocyte globulin (Thymoglobulin, Atgam, Grafalon), alemtuzumab (Campath, Lemtrada), ruxolitinib, itacitinib, and combinations thereof.

10. A method for treating an immune-mediated systemic inflammatory disorder in a subject, comprising administering to the subject a therapeutically effective amount of a GLP-2 analogue or GLP-2 agonist.

11. The method of claim 10, wherein the immune-mediated systemic inflammatory disorder is selected from the group consisting of multiple sclerosis, rheumatoid arthritis, solid organ transplant rejection, autoimmune hepatitis, nonalcoholic steatohepatitis, celiac disease, inflammatory bowel disease, food allergies, and asthma.

12. The method of claim 10, wherein the autoimmune disorder is inflammatory bowel disease (IBD).

13. A method for improving the effect of a cancer treatment in a subject in need thereof, comprising administering to the subject a therapeutically effective amount of a GLP-2 analogue or GLP-2 agonist.

14. The method of claim 13, wherein the administration of a GLP-2 analogue or GLP-2 agonist is after the subject received the cancer treatment regimen.

15. The method of claim 13, wherein the cancer treatment regimen is selected from chemotherapy, radiotherapy, immunotherapy, allogeneic transplant, and combinations thereof.

16. The method of claim 15, wherein the chemotherapy comprises comprising co-administering to the subject a chemotherapy drug selected from the group consisting of cisplatin, temozolomide, doxorubicin, cyclophosphamide, methotrexate, 5-fluorouracil, vinorelbine, docetaxel, bleomycin, vinblastine, dacarbazine, mustine, melphalan, vincristine, procarbazine, prednisolone, etoposide, epirubicin, capecitabine, methotrexate, folinic acid, oxaliplatin, fludarabine, busulfan, clofarabine, and combinations thereof.

17. The method of claim 13, wherein the cancer treatment regimen is allogeneic hematopoietic stem-cell transplantation (HSCT).

18. The method of claim 13, wherein the improvement of effect includes lower gut epithelial toxicity of the cancer treatment.

19. A method for systemically treating an inflammatory condition in a subject caused by solid organ transplant rejection, comprising administering to the subject a therapeutically effective amount of a GLP-2 analogue or GLP-2 agonist.

20. A method for reducing high-dose chemotherapy- and/or radiotherapy-induced GI mucositis, comprising administering to a subject in need thereof an effective amount of a GLP-2 analogue or GLP-2 agonist, wherein the GLP-2 analogue or GLP-2 agonist is administered after completion of the chemotherapy and/or radiotherapy.

21. A method for modulating gut microbiome in a subject in need thereof, comprising administering to the subject a therapeutically effective amount of a GLP-2 analogue or GLP-2 agonist.

22. A method for enhancing the innate immune system in a subject, comprising administering to the subject a therapeutically effective amount of a GLP-2 analogue or GLP-2 agonist.

23. The method of claim 22, wherein the subject has an immune-mediated systemic inflammatory disorder.

24. The method of claim 22, wherein enhancing the innate immune system comprises recovering homeostasis of an innate immune cell.

25. The method of claim 24, the innate immune cell is selected from the group consisting of a macrophage, a dendritic cell, an innate lymphoid cell, and combinations thereof.
